(19) United States
(12) Patent Application Publication
(10) Pub. No.: US 2002/0058076 A1
(43) Pub. Date: May 16, 2002

(54) PROCESS FOR OBTAINING AN ACTIVE PRINCIPLE WITH FIRMING EFFECT TO COUNTER AGING OF THE SKIN, THE OBTAINED FIRMING AGENT AND COMPOSITION USING SUCH AN AGENT

(75) Inventor: Jean-Jacques Paufique, Objat (FR)

Correspondence Address: YOUNG & THOMPSON, 745 SOUTH 23RD STREET 2ND FLOOR, ARLINGTON, VA, 22202

(73) Assignee: S.A. Societe Industrielle Limousine D'application, OBJAT (FR)

(21) Appl. No.: 10/051,175

(22) Filed: Jan. 22, 2002

Related U.S. Application Data
(62) Division of application No. 09/482,885, filed on Jan. 14, 2000.

(30) Foreign Application Priority Data
Dec. 16, 1999 (FR) 99 16098

(51) Int. Cl. A61K 35/78; C12P 21/06

(52) U.S. Cl. 424/750; 435/68.1

(57) ABSTRACT

A process for obtaining an active principle with firming effect for the skin, obtained from wheat protein, provided to counter the aging of the skin includes in succession the following steps:

a) crushing grains of fresh wheat so as to obtain flour;

b) dissolving this fresh wheat flour in water;

c) hydrolyzing this solution in the presence of proteases;

d) effecting a gross separation of the soluble and insoluble phases,

e) concentrating the active fraction; and

f) sterile filtering to retain the microorganisms, yeast and molds as well as the total mesophilic flora.

The present invention provides a process for obtaining an active principle with tensor effect for the skin, obtained from proteins of fresh wheat which permits countering the aging of the skin. The invention also covers the active principle with tensor effect obtained, and the compositions using such an active principle with tensor effect.

The skin is constituted by several layers, the outermost is directly visible and governs the general appearance.

A young skin has a network of microdepressions which are well structured with meshes forming a regular mosaic. The layers are oriented harmoniously in several directions.

This network, upon aging, is destroyed, the meshes enlarge and the layers of a region oriented in a principal direction. The skin becomes wrinkled and wrinkles of various sizes appear.

It is also known that the epidermis loses thickness at the rate of 5 to 7% every ten years with the disappearance of the elastic fibers.

Analysis of the microstructure permits determining the condition of the skin. An analysis of images thus permits qualifying the effects obtained by the different treatments and particularly those of the active principle with tensor effect according to the present invention.

The process of obtaining the active principle with tensor effect according to the present invention and the active principle with tensor effect thus obtained, will now be described in detail.

The different accompanying figures show the different results obtained, and more particularly:

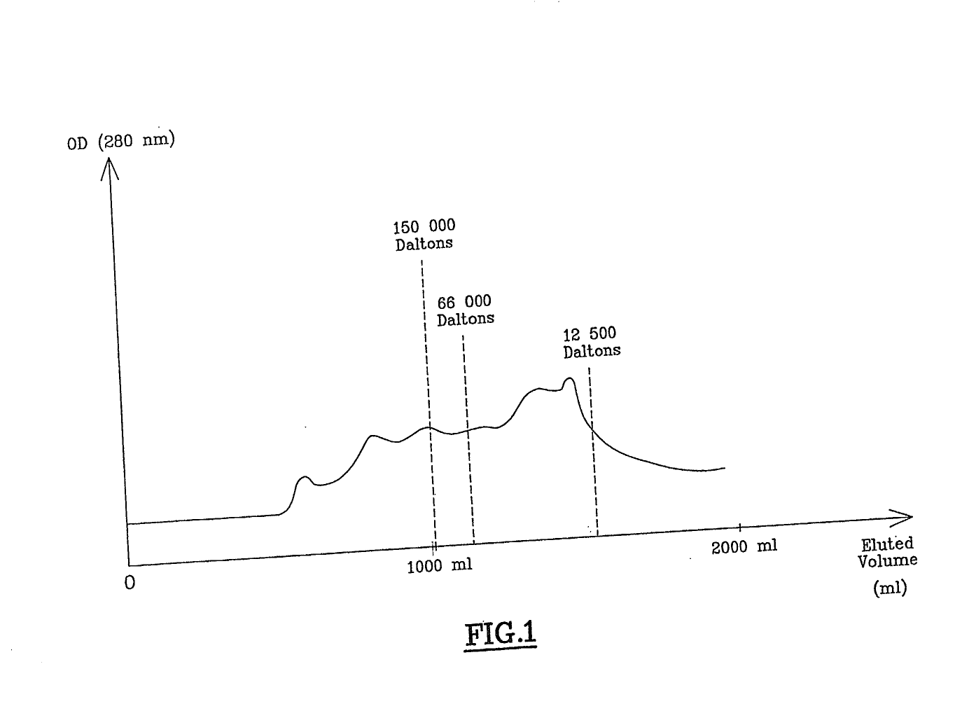

FIG. 1, a chromatogram of the active principle according to the present invention,

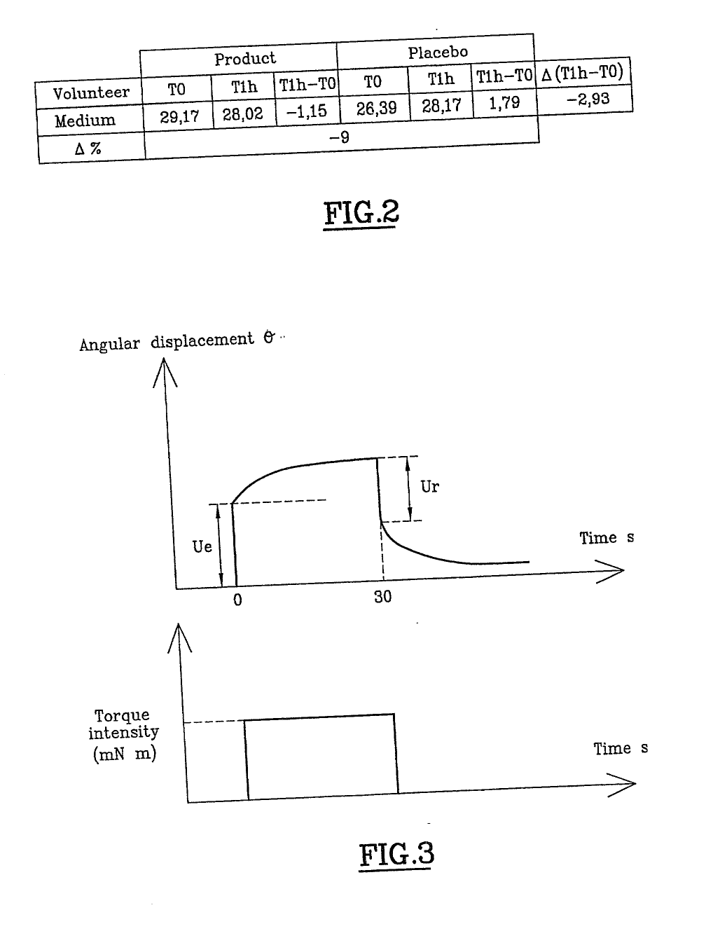

FIG. 2, measurements of roughness of the skin of a panel of volunteers,

FIG. 3, the diagram of angular displacement of a region of the skin subjected to a tortional couple, and

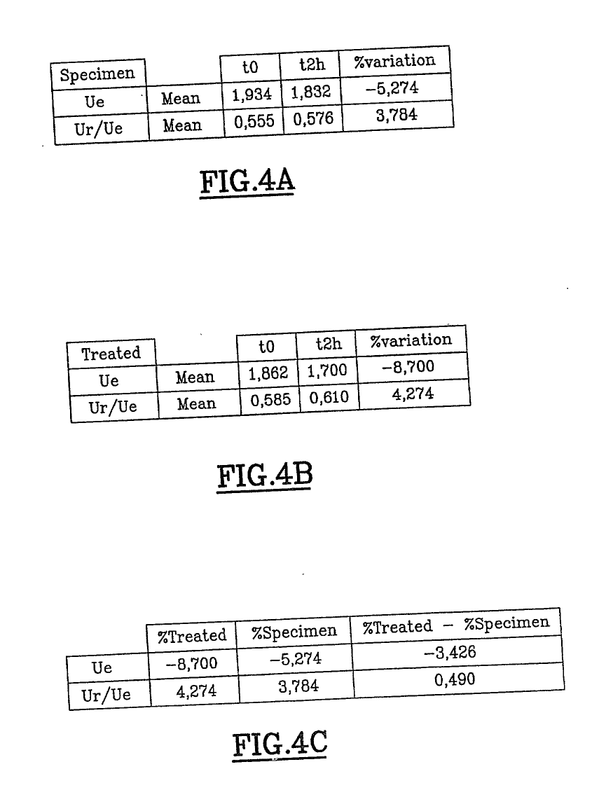

FIGS. 4A, 4B and 4C, the variations of the values of Ue and Ur/Ue, which parameters correspond to the extensibility of the skin and to the cutaneous firmness.

The active principle with tensor effect according to the present invention comprises essentially proteins of high molecular weight extracted from fresh wheat.

The process of extraction consists in a sequence of the following steps:

crushing the grains of fresh wheat,

dissolving the fresh wheat flour thus obtained in water, and preferably at the rate of at least 80 g/l,

hydrolyzing this solution in the presence of proteases, at a pH comprised between 4.0 and 10.0 and a temperature comprised between 35 and 80° C.,

inactivating this hydrolyzed solution,

making a gross separation of the soluble and insoluble phases, by centrifugation, by decantation or by filtration,

concentrating the active fraction, and

filtering under sterile conditions to retain the microorganisms, yeast and molds as well as the total mesophilic flora which must be less than 100 germs/gram.

The obtained extract is characterized by the following data:

1/ Quantity of dry material greater than 50 g/l, comprised between 50 and 200, more particularly between 90 and 110 g/l. This content is obtained by passage through an oven at 105° C. until a constant weight is obtained.

2/ pH comprised between 2.0 and 10, particularly between 6.0 and 9.0, more particularly between 7.0 and 8.0. This value is obtained by the potentiometric method, at ambient temperature.

3/ Quantity of proteins comprised between 50 and 200, more particularly between 70 and 100 g/l. The determination of total nitrogen is obtained by the KJELDAHL method (official method of analysis of the A.O.C. 1975, 12th ed W. Horwitz, N.Y., p. 15-60)

4/ Chromatographic profile:

The molecular mass of the different molecular species is estimated by molecular filtration F.P.L.C. (Fast Protein Liquid Chromatography).

The filtration column, of the type sold under the mark “superdex 75” is graduated with known molecular weight markers, such as:

cytochrome C: 12,500 daltons,

bovine albumin: 66,000 daltons,

dishydrogenated alcohol: 150,000 daltons.

The detection of the eluted components is carried out in ultraviolet at 280 nm.

The results indicated in the table of FIG. 1 are obtained, which augment the characteristics of the active principle obtained.

After characterization of the obtained extract, it is interesting to determine the effects obtained on the skin and to quantify the results.

### I/ Quantification of the Immediate Smoothing Effect

The test consists in quantifying in vivo this immediate smoothing effect in 20 volunteers, against a placebo, one hour after application of a composition in the form of a gel of Carbopol formulated with 5% of the active principle.

An impression of the 20 volunteers is taken before treatment, on a predetermined region of each of the cheeks of each patient. Then, the two produces, active principle and placebo are applied to these regions in the amount of 2 μl/cm². One hour after application, a new impression of the same regions is taken, under constant conditions of temperature and of relative humidity.

Illumination with a shaving light at 35° generates on the surface of the impressions, shadows which are quantified by an image analyzer.

There is thus obtained a data image whose analysis of the levels of gray permits determining an index of roughness which qualifies the relief of the cutaneous surface.

There are then calculated variations of roughness in the two regions treated by the product or by the placebo, before and one hour after application.

There is computed the mean value and there are obtained the results gathered in the table labelled FIG. 2.

The percentage obtained from the means values leads to a decrease of 9% of the roughness and this with 74% of the tested volunteers.

The active principle gives rise to a significant immediate smoothing effect which indicates that the skin has received cutaneous relief (p=0.041).

### II/ Sensorial Evaluation of the Tensor Effect

This test is conducted with the active principle in the amount of 5% in a gel, particularly the one sold under the term Carbopol, against a placebo.

It involved the volunteers evaluating the tensor effect of each of the products when they are applied to a predetermined cutaneous region, where crow's feet appear.

A study of 26 volunteers has shown that the active principle according to the invention has a tensor effect significantly greater than the placebo.

### III/ Quantification of the Tensor Effect by the “Twistometric” Method

This test consists in measuring the angular displacement of the skin under the influence of torsional couple applied parallel to the plane of the skin, which permits determining the state of firmness of the superficial layers of the epidermis.

Two parameters are measured:

Ue, which is the indication of immediate extensibility of the skin, see FIG. 3. This value is defined as the value of the angle of deformation of the skin 50 milliseconds after the beginning of the application of the torsional couple, and

Ur, which is the capacity of the skin to return to its initial position, which parameter is also called recovery.

Ur/Ue corresponds to cutaneous firmness.

Tests were conducted by carrying out on each volunteer a measurement of the zones treated one by one with a cosmetic composition containing 5% of active principle and the other a placebo, immediately before application of the product and 2 hours after application of the product.

There are obtained the results summarized in tables of FIGS. 4A, 4B and 4C.

There will be noted a decrease of the immediate extensibility, of 3.4%, which shows the better reactivity of the skin treated by the composition containing the active principle according to the present invention.

Moreover, cutaneous firmness is improved.

This effect results from the formation of a protein film at the surface of the skin which transmits the tensor effect.

The skin treated with the active principle with tensor effect, more particularly at compositions comprised is between 0.05% and 20% by weight, appears more flexible, more firm, less deformable and the lines and wrinkles disappear.

A fraction of this active principle with tensor effect can also be used for topical application to the skin. Concentrations of active principle can thus be comprised between 0.01% and 50% by weight.

1. A process for firming the skin to counter the effects of aging, comprising applying to the skin of a person in need of the same a cosmetologically effective amount of a composition obtained from wheat proteins by crushing grains of fresh wheat so as to obtain a flour, dissolving this flour in water, hydrolyzing the solution thus obtained in the presence of proteases, separating the soluble and insoluble phases in the hydrolyzed material, and concentrating the soluble phase thereby to concentrate the active principle thus obtained, the molar mass of the different molecular species present having a principal peak at about 15,000 Daltons.

2. A process as claimed in claim 1, wherein said flour is solubilized in an amount of at least 80 g/l of water.

3. A process as claimed in claim 1, wherein the conditions of said hydrolysis are such that the pH is maintained constant between 4.0 and 10.0.

4. A process as claimed in claim 1, wherein the conditions of said hydrolysis are such that the temperature is maintained between 35 and 80° C.
